SEQUENCE LISTING (5 sequences)

SEQ ID NO: 1 - DNA, 2667 bp, Homo sapiens
atggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatga

SEQ ID NO: 2 - protein, 888 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 3 - DNA, 3206 bp, Mouse
gggcagcgggctcggcggggctgcatctaccagcgctgcggggccgcgaacaaaggcgagcagcggaggcgcgagagcaaagtctgaaatggatgttacatgaatcactttaagggctgcgcacaactatgaacgttctgaagccgttttctcactaaagtcactcaagatggatgagtctgccttgttggaccttctggagtgccctgtgtgtctagaacgcctggatgcttccgcaaaggtcttaccctgccagcataccttttgcaaacgctgtttgctggggattgtgggttcccggaatgaactcagatgtcccgaatgccggactcttgttggctctggggtcgacgagctccccagtaacatcctactggtcagacttctggatggcatcaagcagaggccttggaaacccggccctggtgggggcggcgggaccacctgcacaaacacattaagggcgcagggcagcactgtggttaattgtggctcgaaagatctgcagagctcccagtgtggacagcagcctcgggtgcaagcctggagccccccagtgaggggaatacctcagttaccgtgtgccaaagcattatataactacgaaggaaaagagcccggagaccttaagttcagcaaaggcgacaccatcattctgcgccgacaggtggatgagaattggtaccacggggaagtcagcggggtccacggctttttccccactaacttcgtgcagatcatcaaacctttacctcagcccccgcctcagtgcaaagcactttacgactttgaagtgaaagacaaggaagctgacaaagattgccttcccttcgcaaaggacgacgtactgaccgtgatccgcagagtggatgaaaactgggctgaaggaatgctggcagataaaataggaatatttccaatttcatacgtggagtttaactcagctgccaagcagctgatagagtgggataagcctcccgtgccaggagtggacacggcagaatgcccctcagcgacggcgcagagcacctctgcctcaaagcaccccgacaccaagaagaacaccaggaagcgacactccttcacctccctcaccatggccaacaagtcttcccaggggtcccagaaccgccactccatggagatcagccctcctgtgctcatcagttccagcaaccccacagccgcagcccgcatcagcgaactgtccgggctctcctgcagcgccccgtctcaggtccatataagcaccactgggttaattgtgaccccaccccctagcagcccggtgacaactggccctgcgttcacgttcccttcagatgtcccctaccaagctgcccttggaagtatgaatcctccacttcccccaccccctctcctggcggccaccgtactcgcctccaccccgtcaggcgctactgctgctgttgctgctgctgctgccgccgccgccgctgctggaatgggacccaggcctgtgatggggtcctctgaacagattgcacatttacggcctcagactcgtcccagtgtatatgttgctatatatccgtacactccccggaaggaagacgaactggagctgaggaaaggggagatgtttttggtgtttgagcgttgccaggacggctggtacaaagggacatcgatgcataccagcaagataggcgttttccctggcaactatgtggcgcccgtcacaagggcggtgacgaatgcctcccaagctaaagtctctatgtctactgcgggtcaggcaagtcgcggggtgaccatggtcagcccttccactgcaggaggacctacacagaagccccaaggaaacggcgtggccggaaatcccagcgtcgtccccacggctgtggtgtcagcagctcatatccagacaagtcctcaggctaaggtcctgctgcacatgtctgggcagatgacagtcaatcaggcccgcaatgctgtgaggacagttgcagcacatagccaggaacgccccacagcagcagtgactcccatccaggtccagaatgccgcctgccttggtcctgcatccgtgggcctgccccatcattctctggcctcccaacctctgcctccaatggcgggtcctgctgcccacggtgctgccgtcagcatcagtcgaaccaatgcccccatggcctgcgctgcaggggcttctctggcctccccaaatatgaccagtgccatgttggagacagagcccagtggtcgcacagtgaccatcctccctggactccccacatctccagagagtgctgcatcagcgtgtgggaacagttcagctgggaaaccagacaaggacagtaagaaagaaaaaaagggcctactgaagctgctttctggtgcctccaccaaacgcaagccccgagtctcccctccagcatcacctaccctggatgtggagctgggtgctggggaggctcccttgcagggagcagtaggtcctgagctgccgctagggggcagccacggcagagtggggtcatgccccacagatggtgatggtccagtggccgctggaacagcagccctagcccaggatgccttccaccgcaagacaagctccctggactccgcagtgcccattgctccaccacctcgccaggcctgctcctccctgggcccagtcatgaatgaggcccggcctgttgtttgtgaaaggcacagggtggtggtttcctaccctcctcagagtgaggccgaacttgaactcaaggaaggagatattgtgtttgttcataagaaacgagaggacggctggttcaaaggcacgttacagaggaatgggaagactggccttttcccagggagctttgtggaaaacatctgagaagacgggacacggagaaagcttatcatcacaccacgtgtgactaaagagcacaaagcagtttcatagaaagagcacatctgtggacttccagatcttcaagaaccgagcagaagatgggcacctgactccagagccccggcctggttaccccaggggcagagggaaggaggacacacctgtgtgggttccgtctctctgggttctgatgtgtaaagtgtgccttgtaatgtctaatggactttacagataaatgtctttttttttttaagatgtataactaaaatggacaattgtttacaaggcttaactaatttatttgcttttttaaaacttgaactttcttgtaatagcaaat

SEQ ID NO: 4 - protein, 892 aa, Mouse
MDESALLDLLECPVCLERLDASAKVLPCQHTFCKRCLLGIVGSRNELRCPECRTLVGSGVDELPSNILLVRLLDGIKQRPWKPGPGGGGGTTCTNTLRAQGSTVVNCGSKDLQSSQCGQQPRVQAWSPPVRGIPQLPCAKALYNYEGKEPGDLKFSKGDTIILRRQVDENWYHGEVSGVHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDTAECPSATAQSTSASKHPDTKKNTRKRHSFTSLTMANKSSQGSQNRHSMEISPPVLISSSNPTAAARISELSGLSCSAPSQVHISTTGLIVTPPPSSPVTTGPAFTFPSDVPYQAALGSMNPPLPPPPLLAATVLASTPSGATAAVAAAAAAAAAAGMGPRPVMGSSEQIAHLRPQTRPSVYVAIYPYTPRKEDELELRKGEMFLVFERCQDGWYKGTSMHTSKIGVFPGNYVAPVTRAVTNASQAKVSMSTAGQASRGVTMVSPSTAGGPTQKPQGNGVAGNPSVVPTAVVSAAHIQTSPQAKVLLHMSGQMTVNQARNAVRTVAAHSQERPTAAVTPIQVQNAACLGPASVGLPHHSLASQPLPPMAGPAAHGAAVSISRTNAPMACAAGASLASPNMTSAMLETEPSGRTVTILPGLPTSPESAASACGNSSAGKPDKDSKKEKKGLLKLLSGASTKRKPRVSPPASPTLDVELGAGEAPLQGAVGPELPLGGSHGRVGSCPTDGDGPVAAGTAALAQDAFHRKTSSLDSAVPIAPPPRQACSSLGPVMNEARPVVCERHRVVVSYPPQSEAELELKEGDIVFVHKKREDGWFKGTLQRNGKTGLFPGSFVENI

SEQ ID NO: 5 - protein, 9 aa, Artificial Sequence
  nuclear localization sequence
PPKKKRKVA

(19) United States
(12) Patent Application Publication
(10) Pub. No.: US 2005/0181453 A1
(43) Pub. Date: Aug. 18, 2005

(54) Ubiquitin-based protein interaction assays and related compositions

(75) Inventors: Shmuel Tuvia, Netanya (IL); Daniel N. Taglicht, Lapid (IL)

Correspondence Address: FISH & NEAVE IP GROUP;ROPES & GRAY LLP, ONE INTERNATIONAL PLACE, BOSTON, MA, 02110-2624, US

(73) Assignees: Proteologics, Inc., Rehovot (IL); Proteologics, Ltd., Rehovot (IL)

(21) Appl. No.: 10/979,766

(22) Filed: Nov. 1, 2004

Related U.S. Application Data
(63) Continuation-in-part of application No. 10/977,350, filed on Oct. 28, 2004.
(60) Provisional application No. 60/516,152, filed on Oct. 31, 2003.

(51) Int. Cl. G01N 33/53; C12Q 1/37

(52) U.S. Cl. 435/7.1; 435/23

(57) ABSTRACT

The invention provides, inter alia, methods for identifying substrates for E3 proteins that mediate ligation of ubiquitin or ubiquitin-like proteins.

### BACKGROUND OF THE INVENTION

The ubiquitin-mediated proteolysis system is the major pathway for the selective, controlled degradation of intracellular proteins in eukaryotic cells. Ubiquitin modification of a variety of protein targets within the cell is important in a number of basic cellular functions such as regulation of gene expression, regulation of the cell-cycle, modification of cell surface receptors, biogenesis of ribosomes, and DNA repair, and therefore, the ubiquitin system has been implicated in the pathogenesis of numerous disease states, including oncogenesis, inflammation, viral infection, CNS disorders, and metabolic dysfunction.

One major function of the ubiquitin-mediated system is to control the half-lives of cellular proteins. The half-life of different proteins can range from a few minutes to several days, and can vary considerably depending on the cell-type, nutritional and environmental conditions, as well as the stage of the cell-cycle. Targeted proteins undergoing selective degradation, presumably through the actions of a ubiquitin-dependent proteosome, are covalently tagged with ubiquitin through the formation of an isopeptide bond between the c-terminal glycyl residue of ubiquitin and a specific lysyl residue in the substrate protein. This process is catalyzed by a ubiquitin-activating enzyme (E1) and a ubiquitin-conjugating enzyme (E2), and may also require auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. In an initial ATP-dependent step, a thioester is formed between the c-terminus of ubiquitin and an internal cysteine residue of an E1 enzyme. Activated ubiquitin is then transferred to a specific cysteine on one of several E2 enzymes. Finally, these E2 enzymes donate ubiquitin to protein substrates. Substrates are recognized either directly by ubiquitin-conjugated enzymes or by associated substrate recognition proteins, the E3 proteins, also known as ubiquitin ligases.

In addition to the 76-amino acid ubiquitin, there is a family of ubiquitin-like protein modifiers that are low molecular weight polypeptides (76-165 amino acids) and share between 10% and 55% sequence identity to ubiquitin. See, e.g., Wong et al., Drug Discovery in the Ubiquitin Regulatory Pathway, DDT 8(16): 746-54, August 2003; Schwartz & Hochstrasser, A Superfamily of Protein Tags: Ubiquitin, SUMO and Related Modifiers, Trends Biochem. Sci. 28(6): 321-28, June 2003. Although ubiquitin and each ubiquitin-like protein modifier direct distinct sets of biological consequences and each requires distinct conjugation and deconjugation machinery, they share a similar cascade mechanism involving an activating enzyme (E1), a conjugating enzyme (E2), and perhaps an auxiliary substrate recognition protein (E3, also termed ligase).

Genome mining efforts have identified at least 530 human genes that encode enzymes responsible for conjugation and deconjugation of ubiquitin or ubiquitin-like protein modifiers. See, e.g., Wong et al., supra. A multitude of E3s reflect their roles as specificity determinants; as a modular system, each E2-E3 pair appears to recognize a distinct set of cellular substrates. For example, the same E2 in conjunction with different E3s may recognize distinct substrates. The human genome encodes 391 potential E3s, as defined by the presence of HECT, RING finger, PHD or U-box domains. Wong et al., supra. The domains mediate the interaction of the E3 with the E2. E3s encompass a broad spectrum of molecular architectures ranging from large multimeric complexes (e.g., anaphase promoting complex or APC), in which E2 binding, substrate recognition, and regulatory functions reside in separate subunits, to relatively simple single component enzyme (e.g., murine double minute or MDM2) in which all necessary functions are incorporated into one polypeptide.

As important regulatory mechanisms underlying diverse biological pathways, ubiquitin and ubiquitin-like protein modification systems present novel targets in the treatment of multiple diseases. Accordingly, it is an object of the invention to delineate these protein modification systems, for example, by identifying novel protein substrates for ubiquitination (or ubiquitination, an equivalent term used in the art) or modifications by ubiquitin-like protein modifiers such as sumoylation.

### BRIEF SUMMARY OF THE INVENTION

Accordingly, in certain aspects, the present invention provides cell-based systems for determining whether a first polypeptide mediates the attachment of a ubiquitin or ubiquitin-like protein to a prey polypeptide. As will be apparent from the description, methods disclosed herein may be adapted for evaluating specific interactions between a known first polypeptide (e.g, an E3 protein) and a known prey polypeptide (e.g., a known substrate for the E3 protein) and may also be adapted for identifying previously unknown substrates for E1, E2 or E3 enzymes or for identifying previously unknown E1, E2 or E3 enzymes that act upon a particular substrate of interest.

In certain embodiments, a method for determining whether a first polypeptide mediates the attachment of a ubiquitin or ubiquitin-like protein to a prey polypeptide comprises providing a host cell comprising: a) a first nucleic acid encoding said first polypeptide, b) a second nucleic acid encoding a bait fusion protein comprising a bait polypeptide fused to a first output-inducing polypeptide; and c) a third nucleic acid encoding a prey fusion protein comprising a prey polypeptide fused to a second output-inducing polypeptide, wherein said bait polypeptide comprises a ubiquitin or ubiquitin-like protein, or a fragment thereof sufficient to achieve covalent attachment to a suitable prey polypeptide. Generally, the first polypeptide will be selected so as to be a polypeptide that mediates covalent attachment of the bait polypeptide to a prey polypeptide that is a protein substrate of said first polypeptide. For example, the first polypeptide may be an E3 or E2 enzyme or an polypeptide suspected of being an E2 or E3 enzyme. The first polypeptide may be a particular, pre-selected protein, or it may be, for example, a representative from a library of E2 or E3 enzymes. Likewise, the prey polypeptide may be a particular pre-selected protein that is, for example, known to be covalently modified by a ubiquitin or ubiquitin-like moiety. A prey polypeptide may also be a representative from a library of prey polypeptides of any sort. The first and second output-inducing polypeptides will generally be selected such that they generate an output signal when the bait fusion protein interacts with the prey fusion protein. Generally, greater physical proximity of the first and second output-inducing polypeptides will create a greater output signal. The detection of an output signal will indicate that said first polypeptide mediates the attachment of a ubiquitin or ubiquitin-like protein to said prey polypeptide, although subsequent control experiments may be desirable to validate the result. For example, a method may further comprise determining whether the presence of said output signal is dependent on the presence of said first protein in said host cell, wherein the dependency indicates said first polypeptide mediates the attachment of a ubiquitin or ubiquitin-like protein to said prey polypeptide. In a two-hybrid-like variation, the method employs a first output-inducing peptide that comprises a DNA binding domain of a transcriptional activator and a second output-inducing peptide comprises an activation domain of a transcriptional activator. The output signal may be the expression of a reporter gene that is activated by said transcriptional activator. The host cell may further comprise an additional nucleic acid encoding an exogenous E1 or E2 protein, as desirable to facilitate the activation of an E2 or E3 protein used in the method.

In certain aspects the present invention provides methods and systems for identifying novel protein substrates for ubiquitination or modifications by ubiquitin-like protein modifiers. Certain methods disclosed herein utilize the ability of an E3 protein to mediate the formation of a covalent bond between a ubiquitin- (or a ubiquitin like protein) containing fusion protein and a substrate-containing fusion protein to identify substrate. Each fusion protein may be designed to contain an output-generating domain such that, upon bond formation between ubiquitin and substrate, the two output domains are brought into close proximity and an output signal is generated. In certain preferred embodiments, methods disclosed herein employ a library of nucleic acids to screen for candidate E3 substrates, or other highly parallel systems for identifying E3 substrates.

In certain embodiments, the invention provides a method for identifying a protein substrate for an E3 protein, comprising: i. providing a host cell comprising: a) a first nucleic acid encoding said E3 protein; b) a second nucleic acid encoding a bait fusion protein comprising a bait polypeptide sequence fused to a first output-inducing peptide sequence; and c) a third nucleic acid encoding a prey fusion protein comprising a prey polypeptide sequence fused to a second output-inducing peptide sequence, wherein said E3 uses said bait polypeptide as a protein modifier, and wherein physical proximity of said first and second output-inducing peptides induces an output signal; and ii. detecting said output signal; wherein the presence of said output signal indicates said prey polypeptide comprises a candidate protein substrate for said E3 protein. While the prey polypeptide may be a representative of essentially any library, in certain embodiments, the prey polypeptide will be selected as a protein that is known to interact with the E3 protein, and the method may be used to distinguish general E3 interacting proteins from those that interact with the E3 protein as substrates thereof. This may be accomplished by, for example, determining whether the presence of said output signal is dependent on the presence of said candidate E3 protein in said host cell, wherein the dependency indicates that said candidate E3 protein acts as an E3 protein with respect to the prey polypeptide.

In certain embodiments, the invention provides a method for identifying a candidate E3 protein that acts on a protein substrate, comprising: i. providing a host cell comprising: a) a first nucleic acid encoding said candidate E3 protein, b) a second nucleic acid encoding a bait fusion protein comprising a bait polypeptide fused to a first output-inducing polypeptide; and c) a third nucleic acid encoding a prey fusion protein comprising a prey polypeptide substrate fused to a second output-inducing polypeptide, wherein said prey polypeptide substrate is known to be the substrate of an E3 protein, and wherein physical proximity of said first and second output-inducing polypeptides induces an output signal; and ii. detecting said output signal; wherein the presence of said output signal indicates that said candidate E3 protein acts as an E3 protein with respect to the prey polypeptide.

In certain embodiments, the invention provides a method for identifying a protein substrate for an E2 protein, comprising: i. providing a host cell comprising: a) a first nucleic acid encoding said E2 protein, b) a second nucleic acid encoding a bait fusion protein comprising a bait polypeptide fused to a first output-inducing polypeptide; and c) a third nucleic acid encoding a prey fusion protein comprising a prey E3 polypeptide fused to a second output-inducing polypeptide, wherein physical proximity of said first and second output-inducing polypeptides induces an output signal; and ii. detecting said output signal; wherein the presence of said output signal indicates that said prey E3 polypeptide comprises a candidate protein substrate for said E2 protein.

In certain embodiments, the invention provides a method for identifying a candidate E2 protein that acts on an E3 protein substrate, comprising: i. providing a host cell comprising: a) a first nucleic acid encoding said candidate E2 protein, b) a second nucleic acid encoding a bait fusion protein comprising a bait polypeptide fused to a first output-inducing polypeptide; and c) a third nucleic acid encoding a prey fusion protein comprising a prey E3 polypeptide substrate fused to a second output-inducing polypeptide, wherein physical proximity of said first and second output-inducing polypeptides induces an output signal; and ii. detecting said output signal; wherein the presence of said output signal indicates that said candidate E2 protein acts as an E2 protein with respect to the prey E3 polypeptide.

In a preferred embodiment, the method further provides the step of determining whether the presence of the output signal is dependent on the presence of an E3 protein. Where the output signal is determined to be dependent on the presence of an E3 protein, the prey polypeptide comprises a desired protein substrate for the E3 protein.

In one embodiment, the bait polypeptide comprises ubiquitin (“Ub”) or a fragment of ubiquitin that can be used by an E3 protein to modify a protein substrate. In another embodiment, the bait polypeptide comprises a ubiquitin-like protein modifier (“Ub1”) or a fragment of a Ub1 that can be used by an E3 protein to modify a protein substrate.

In another embodiment, the output signal is the expression of a reporter gene, which is operably linked to a transcriptional regulatory element and of which transcription is activated by the physical proximity of a DNA-binding domain (“DBD”) recognizing the transcriptional regulatory element and a transcription activation domain (“AD”). In a preferred embodiment, the bait fusion protein comprises DBD as the first output-inducing peptide, and the prey fusion protein comprises AD as the second output-inducing peptide.

In another embodiment, the reporter gene is an endogenous gene to the host cell. For example, the reporter gene may β-galactosidase in a yeast host cell.

In another embodiment, the reporter gene may be exogenous to the host cell. For example, the reporter gene may be introduced to the host cell via an expression construct, such as the alkaline phosphatase gene in an mammalian cell expression construct, e.g., pG5SEAP (BD Biosciences, BD MATCHMAKER™ Mammalian Two-Hybrid Kit).

In another embodiment, expression of the E3 or E2 protein is controlled by an inducible promoter. For example, a nucleic acid encoding an E2 or E3 protein may be part of an expression construct comprising an inducible promoter which is operably linked to the coding sequence. The E2 or E3 coding sequence may also be cloned into the same expression construct comprising the bait fusion protein coding sequence. In this embodiment, expression of the bait fusion protein is controlled a promoter distinct from the inducible promoter controlling the expression of E2 or E3.

In another embodiment, the output signal is a change in fluorescence. In a preferred embodiment, each output-inducing peptide comprises a fluorescent protein of a distinct color, and the desired output signal is a change in fluorescence from a single color to both colors from the first and second output-inducing peptides. For example, one output-inducing peptide may comprise a green fluorescence protein (“GFP”), and the other output-inducing peptide may comprise a variant GFP or a blue fluorescent protein (“BFP”) that has spectral characteristics distinct from the other GFP. It is also possible to use the same fluorescent protein as the output-inducing peptides, wherein amplified or cumulative fluorescent signals would be detected as a change in fluorescence.

In another embodiment, exogenous E1 and E2 proteins are introduced to the host cell to form a complete Ub or Ub1-modification system. In a preferred embodiment, E1 and E2 are introduced to the host cell via expression constructs that may comprise inducible promoters to control the expression of E1 or E2.

As will be appreciated by one of ordinary skill in the art, a method of the present invention may not rely on the use of fusion proteins comprising output-inducing peptides. Described below are methods based on technologies such as phage display, wherein Ub or Ub1 without fusing to another peptide will be used together with a ubiquitination machinery to screening for protein substrates that will be modified with the Ub or Ub1 by the particular ubiquitination machinery.

Another aspect of the invention provides a kit for detecting a protein substrate for an E3, said kit comprising: i. a first expression construct comprising a coding sequence for a first output-inducing peptide and a ligation site flanking an end of said first output-inducing peptide coding sequence for ligating a coding sequence of a bait polypeptide sequence in frame with said first output-inducing peptide coding sequence to produce a bait fusion protein, said first expression construct operably linked to a first transcriptional regulatory element; ii. a second expression construct comprising a coding sequence for a second output-inducing peptide and a ligation site flanking an end of said second output-inducing peptide coding sequence for ligating a coding sequence of a prey polypeptide sequence in frame with said output-inducing peptide coding sequence to produce a prey fusion protein, said second expression construct operably linked to a second transcriptional regulatory element; and iii. a nucleic acid comprising a coding sequence for said E3 operably linked to a third transcriptional regulatory element.

In a preferred embodiment, the E3-encoding nucleic acid sequence is part of the first expression construct. In a preferred embodiment, the first expression construct comprises an inducible promoter controlling the expression of the E3-encoding nucleic acid sequence.

In another embodiment, the E3-encoding nucleic acid sequence is part of a third expression construct. In a preferred embodiment, the third expression construct comprises an inducible promoter which controls the expression of the E3-encoding sequence.

In another embodiment, the kit further comprises a reporter gene construct, and expression of the reporter gene is activated by the physical proximity of the first and second output-inducing peptides.

Another aspect of the present invention provides a host cell comprising: i. a first nucleic acid encoding an E3 protein, ii. a second nucleic acid encoding a bait fusion protein comprising a bait polypeptide sequence fused to a first output-inducing peptide; and iii. a third nucleic acid encoding a prey fusion protein comprising a prey polypeptide sequence fused to a second output-inducing peptide, wherein said E3 protein uses said bait polypeptide as a protein modifier, and wherein physical proximity of said first and second output-inducing peptides induces an output signal.

In another embodiment, the host cell further comprises an expression construct that introduce exogenous E1 or E2 or both into the host cell.

Another aspect of the present invention provides a host cell comprising: i. a first nucleic acid encoding an E2 protein, ii. a second nucleic acid encoding a bait fusion protein comprising a bait polypeptide sequence fused to a first output-inducing peptide; and iii. a third nucleic acid encoding a prey fusion protein comprising a prey E3 polypeptide sequence fused to a second output-inducing peptide, wherein physical proximity of said first and second output-inducing peptides induces an output signal.

As will be recognized by a skilled artisan, the same methods and systems described above can be adapted to identify protein substrates for an E2 protein, by converting the first nucleic acid to comprise a coding sequence for an E2 protein. In some embodiments, exogenous E3 may be used, for example, by way of an expression construct. In some embodiments, exogenous E1 may be used, for example, by way of expression construct. In some embodiments, endogenous E1 or E3 or both may complement the subject E2 protein to form a complete Ub or Ub1-modification system (also termed a ubiquitination machinery).

Another aspect of invention relates to uses of and compositions comprising an identified protein substrate for E2 or E3. For example, an identified protein substrate by the present invention may be a useful drug target, and therefore, screening methods and useful compositions relating to the identified protein substrate can be developed, as will be appreciated by a skilled artisan.

### BRIEF DESCRIPTION OF THE DRAWINGS

FIG. 1 is a schematic view of certain embodiments: A ubiquitin/E3-based yeast two-hybrid screening system. Both the E3 and the prey may be defined, known proteins. In addition, the E3 protein may be a known E3 protein, while the prey is varied from cell to cell to allow screening for prey proteins that are substrates for the E3 protein. In a further embodiment, the prey protein may be known, while the E3 protein is varied from cell to cell to allow screening for E3 proteins that have the known prey protein as a substrate. The E3 may be replaced with an E2 protein, in which case the prey protein will generally be an E3 protein. In this manner, the interactions between E2 proteins and their substrates (usually E3 proteins) may be probed.

FIG. 2 shows the human POSH (an exemplary E3) coding nucleic acid sequence.

FIG. 3 shows the human POSH (an exemplary E3) amino acid sequence.

FIG. 4 shows the murine POSH coding nucleic acid sequence and amino acid sequence.

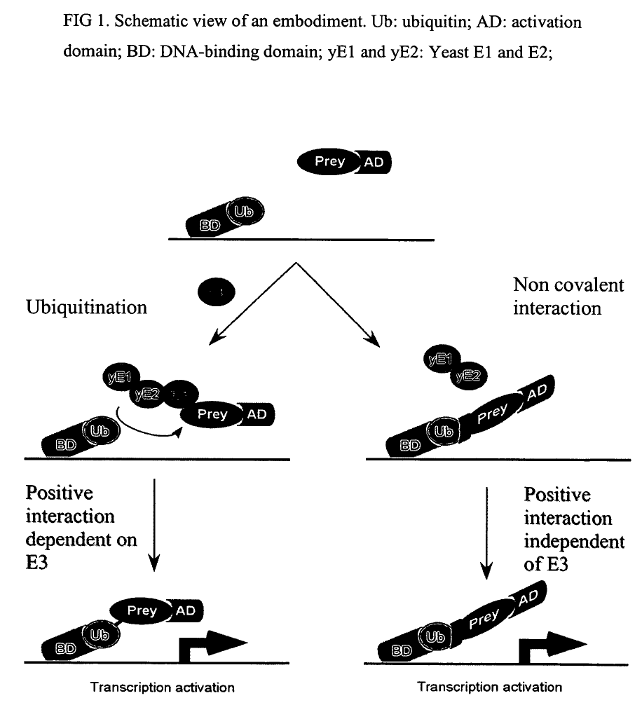

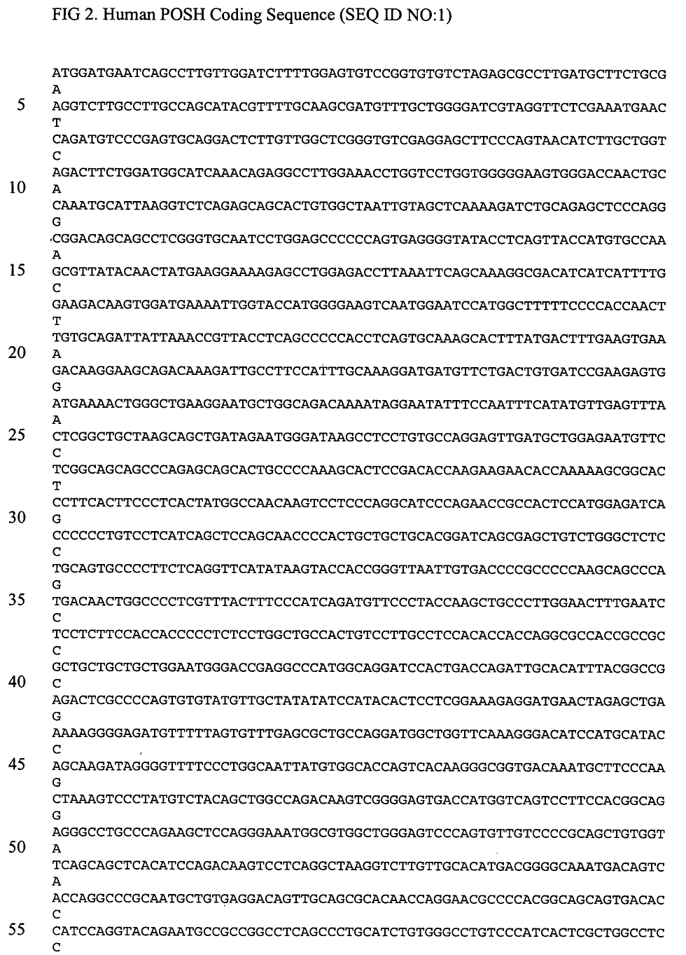

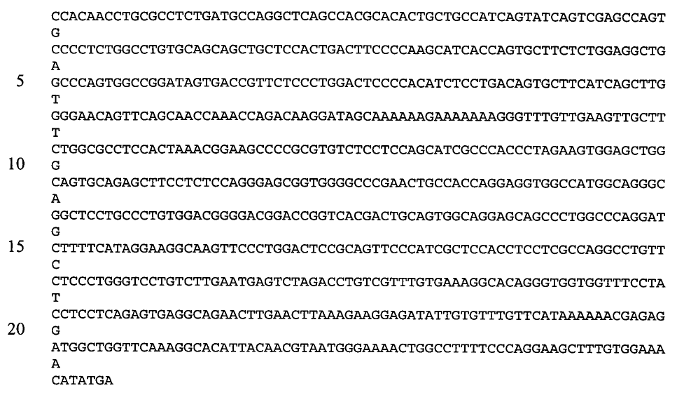

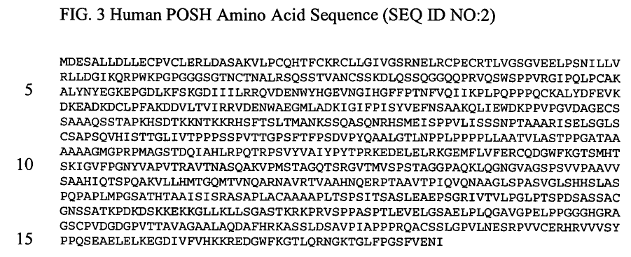

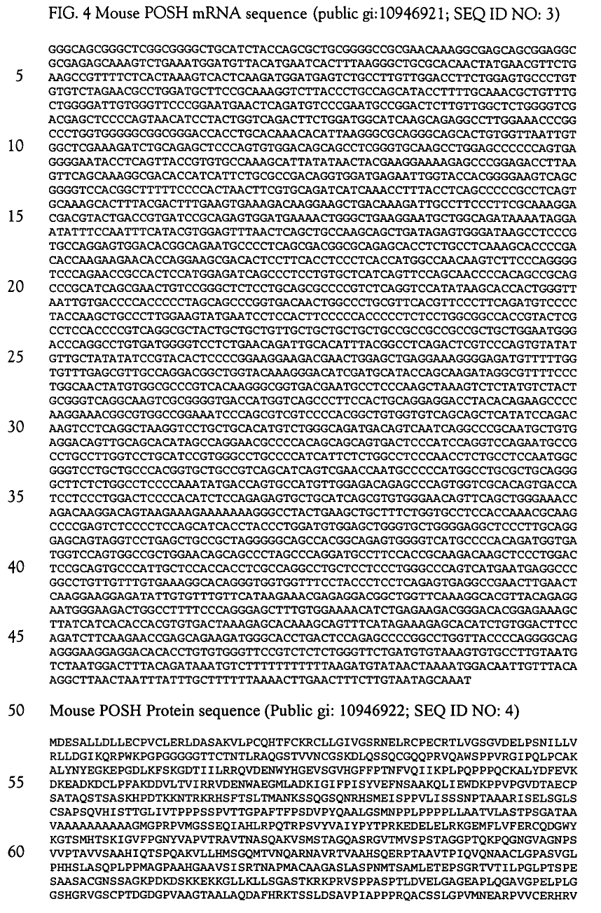

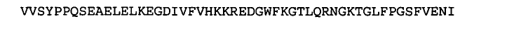

### DETAILED DESCRIPTION OF THE INVENTION

In certain aspects, the present invention provides methods and systems relating to ubiquitin and ubiquitin-like protein modification systems. A ubiquitinated protein substrate is a protein complex comprising ubiquitin covalently attached to the protein substrate. Therefore, the invention provides methods and systems that utilize a ubiquitination machinery and identify specific protein substrates that are ubiquitinated by the machinery, and methods and systems are based on the ubiquitination machinery- or E3-mediated protein-protein interaction between ubiquitin (or another protein modifier) and its protein substrate. Likewise, methods described herein may be used to analyze the interaction between a known E3 and a known substrate, or to identify an E3 for a protein of interest. It is noted that the E3-mediated protein-protein interaction described herein may be distinct from a simple tripartite or ternary protein complex in that E3, acting as an enzyme, catalyzes the protein-protein interaction which leads to a ubiquitinated (or similarly modified) protein substrate. Certain methods and systems described herein are further suitable to conduct high throughput screening, for example, to identify protein substrates subject to ubiquitination or other protein modification.

Naturally occurring ubiquitin, or “Ub,” as used herein refers to an abundant 76 amino acid residue polypeptide that is found in most, if not all, eukaryotic cells. The Ub polypeptide is characterized by a carboxy-terminal glycine residue that is activated by ATP to a high-energy thiol-ester intermediate in a reaction catalyzed by a Ub-activating enzyme (E1). The activated Ub is transferred to a substrate polypeptide via an isopeptide bond between the activated carboxy-terminus of Ub and the epsilon-amino group of a lysine residue(s) in the protein substrate. This transfer requires the action of Ub conjugating enzymes such as E2 and, in some instances, auxiliary substrate recognition or Ub ligase (E3) activities. The Ub-modified substrate is thereby altered in biological function, and, in some instances, becomes a substrate for components of the Ub-dependent proteolytic machinery which includes both Ub isopeptidase enzymes as well as proteolytic proteins which are subunits of the proteasome. As used herein, the term “ubiquitin” or Ub includes within its scope all known as well as unidentified eukaryotic Ub homologs of vertebrate or invertebrate origin. Examples of Ub polypeptides as referred to herein include the human Ub polypeptide which is encoded by the human Ub encoding nucleic acid sequence (GenBank Accession Numbers: U49869, X04803) as well as all equivalents. Another example of a Ub polypeptide as referred to herein is murine Ub which is encoded by the murine Ub nucleic acid coding sequence (GenBank Accession Number: X51730).

The term “ubiquitin-like,” or “Ub1,” protein modifiers as used herein refer to the group of small proteins that are subject to a conjugation machinery similar to ubiquitination. For example, a Ub1 protein modifier can be NEDD8, ISG15, SUMO1, SUMO2, SUMO3, APG12, APG8, as listed in Wong et al., supra, or another Ub1 to be identified. An example of a Ub1 polypeptide as referred to herein is murine SUMO1 (also termed GMP1, Pic1, SMTP3, Smt3C, sentrin) which is encoded by the murine encoding nucleic acid sequence (GenBank Accession Number: NM_{—}009460).

The present invention also contemplates the use of Ub or Ub1 fragments that are sufficient for the Ub conjugation or ubiquitination machinery.

The term “Ub or Ub1 conjugation machinery” or “ubiquitination machinery” as used herein refers to a group of proteins which function in the ATP-dependent activation and transfer of Ub or Ub1 to substrate proteins. The term thus encompasses: E1 enzymes, which transform the carboxy-terminal glycine of Ub or Ub1 into a high energy thiol intermediate by an ATP-dependent reaction; E2 enzymes (the LTBC genes), which transform the E1-S-Ub/Ub1 activated conjugate into an E2S-Ub/Ub1 intermediate which acts as a Ub or Ub1 donor to a substrate, another Ub moiety (in a poly-ubiquitination reaction), or an E3; and the E3 enzymes which facilitate the transfer of an activated Ub or Ub1 molecule from an E2 to a substrate molecule or to another Ub or Ub1 moiety as part of a polyubiquitin chain. The term “Ub or Ub1 conjugation machinery” or ubiquitination machinery as used herein, is further meant to include all known members of these groups as well as those members which have yet to be discovered or characterized but which are sufficiently related by homology to known Ub or Ub1 conjugation enzymes so as to allow an individual skilled in the art to readily identify it as a member of this group. The term as used herein is meant to include novel Ub activating enzymes (E2s) which have yet to be discovered as well as those which function in the activation and conjugation of Ub1 or Ub-related polypeptides to their substrates and to poly-Ub1 or poly-Ub-related protein chains.

Essentially any E3 may be used in methods and systems disclosed herein. For example, Wong et al. discloses four subclasses of E3s: RING, PHD, HECT, and U-box. The RING subclass comprises 439 isoforms (e.g., alternative splicing variants), the PHD subclass 137 isoforms, the HECT subclass 43 isoforms, and the U-box subclass 13 isoforms. Yet other new E3 proteins or isoforms may be discovered. As used herein, the term “E3” or “E3 protein” is intended to encompass any portion of an E3 that is sufficient to mediate ubiquitination of a substrate protein. An E3 may also comprise more than one polypeptide or fragments of polypeptides.

An example of an E3 for use in the methods and systems of the invention is POSH (Plenty Of SH3 domains) nucleic acid sequences and proteins encoded thereby. POSH comprises a RING domain and undergoes a self-mediated ubiquitination. POSH proteins play a role in viral maturation, protein trafficking and other significant biological processes. For example, POSH may act in the assembly or trafficking of complexes that mediate viral release.

“Homology” or “identity” or “similarity” refers to sequence similarity between two peptides or between two nucleic acid molecules. Homology can be determined by comparing a position in each sequence which may be aligned for purposes of comparison. Preferably, such comparisons will be made using the well-known BLAST algorithm. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “nonhomologous” sequence shares less than 40% identity, though preferably less than 25% identity, with one of the E3 sequences of the present invention.

It will be generally appreciated that, under certain circumstances, it may be advantageous to provide homologs of an E3 or Ub or Ub1 of the invention. For example, such homologs may be useful when, e.g., the E3 or Ub or Ub1 also comprises an undesirable biological activity to a host cell of the invention. Thus, an E3 or Ub or Ub1 derived from the nonnaturally occurring homologs may be used to practice the present invention with fewer side effects relative to an E3 or Ub or Ub1 derived from the naturally occurring polypeptides. Accordingly, the terms “E3,” “Ub,” and “Ub1,” are intended to encompass such homologs thereof.

Homologs of each of the subject subunit polypeptides can be generated by mutagenesis, such as by discrete point mutation(s), or by truncation. WO0022110, incorporated in full by reference herein, describes various methods to create polypeptide homologs.

The terms “protein,” “polypeptide” and “peptide” are used interchangeably herein when referring to a gene product. The terms refer to polymers of amino acid of any length. The polymer may be linear or branched, it may comprise modified amino acids, and it may be interrupted by non-amino acids. It also may be modified naturally or by intervention, for example, disulfide bond formation, glycosylation, myristylation, acetylation, alkylation, phosphorylation or dephosphorylation. Also included within the definition are polypeptides containing one or more analogs of an amino acid (including, for example, unnatural amino acids) as well as other modifications known in the art.

As used herein, the term “nucleic acid” refers to polynucleotides such as DNA, and, where appropriate, ribonucleic acid (RNA). The term should also be understood to include analogs of either RNA or DNA made from nucleotide analogs, and, as applicable to the embodiment being described, single (sense or antisense) and double-stranded polynucleotides.

As used herein, the term “promoter” means a DNA sequence that regulates expression of a selected DNA sequence operably linked to the promoter, and which effects expression of the selected DNA sequence in cells. A “promoter” generally is a DNA regulatory region capable of binding RNA polymerase in a cell and initiating transcription of a coding sequence. For example, the promoter sequence may be bounded at its 3′ terminus by the transcription initiation site and extend upstream (5′ direction) to include the minimum number of bases or elements necessary to initiate transcription at levels detectable above background. Within the promoter sequence may be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

The term “promoter” as used herein encompasses “cell specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled). For example, the Met25 promoter present in the pBridge vector (BD Biosciences Catalog #6184-1, a yeast expression vector) is an exemplary inducible promoter in response to methionine levels in the medium: it is repressed in presence of 1 mM methionine and expressed in the absence of methionine. Therefore, a nucleic acid coding sequence, e.g., encoding an E3, operably linked to the Met25 promoter would not be expressed when the yeast cell (transfected with the pBridge vector comprising the coding sequence) the is exposed to the medium containing 1 mM methionine, whereas said coding sequence will be expressed when the yeast cell grows in the medium without methionine.

A “vector” is a replicon, such as plasmid, phage or cosmid, to which another DNA segment may be attached. The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A DNA or nucleic acid “coding sequence” is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence may be located 3′ of the coding sequence.

Nucleic acid or DNA “regulatory sequences” or “regulatory elements,” as used herein, are transcriptional and translational control sequences, such as promoters, enhancers, polyadenylation signals, terminators, and the like, that provide for and/or regulate expression of a coding sequence in a host cell.

Regulatory sequences for directing expression of the instant fusion proteins are art-recognized and may be selected by a number of well understood criteria. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding the fusion proteins of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the promoter for 3-phosphoglycerate kinase or other glycolytic enzymes, the promoters of acid phosphatase, e.g., Pho5, and the promoters of the yeast α-mating factors and other sequences known to control the expression of genes of prokaryotic or eukaryotic cells or their viruses, and various combinations thereof. It should be understood that the design of the expression vector may depend on such factors as the choice of the host cell to be transformed. Moreover, the vector's copy number, the ability to control that copy number and the expression of any other protein encoded by the vector, such as antibiotic markers, should also be considered.

The invention also provides nucleic acids encoding fusion proteins comprising output-inducing peptides, of which physical proximity induces a detectable signal.

Also provided are vector and other nucleic acid constructs comprising the subject nucleic acids, where such constructs may be used for a number of applications, including propagation, protein production, etc. Viral and non-viral vectors may be prepared and used, including plasmids. The choice of vector will depend on the type of cell in which propagation is desired and the purpose of propagation. Certain vectors are useful for amplifying and making large amounts of the desired DNA sequence. Other vectors are suitable for expression in cells in culture. Still, other vectors are suitable for transfer and expression in cells in a whole animal. The choice of appropriate vector is well within the skill of the art, and many such vectors are available commercially. Constructs may be prepared using any available technique. For example, the partial or full-length polynucleotide may be inserted into a vector typically by means of DNA ligase attachment to a cleaved restriction enzyme site in the vector. Alternatively, the desired nucleotide sequence can be inserted by homologous recombination in vivo, typically by attaching regions of homology to the vector on the flanks of the desired nucleotide sequence. Regions of homology may be added by ligation of oligonucleotides, or by polymerase chain reaction using primers comprising both the region of homology and a portion of the desired nucleotide sequence, for example.

Also provided are expression constructs and vectors that find use in, among other applications, the synthesis of the subject proteins, including E3 polypeptide, bait polypeptide and prey polypeptide and fusion proteins thereof. For expression, an expression construct or vector is introduced to any compatible host cell, including, for example, bacterial, yeast, insect, amphibian and mammalian cells. Examples of such vectors and host cells are described in U.S. Pat. No. 5,654,173. In the expression vector, a subject polynucleotide, e.g., a bait fusion protein or a prey fusion protein, is linked to a regulatory sequence as appropriate to obtain the desired expression properties. These regulatory sequences can include promoters (attached either at the 5′ end of the sense strand or at the 3′ end of the antisense strand), enhancers, terminators, operators, repressors and inducers. The promoters can be regulated or constitutive. In some situations it may be desirable to use conditionally active promoters, such as tissue-specific or developmental stage-specific promoters. These are linked to the desired nucleotide sequences using the techniques described above for linkage to vectors.

An expression vector will generally comprise a transcriptional and translational initiation region, which may be inducible or constitutive, where the coding region is operably linked under the transcriptional control of the transcriptional initiation region, and a transcriptional and translational termination region. These control regions may be native to the subject species from which the subject nucleic acid is obtained, or may be derived from exogenous sources.

Expression vectors generally have convenient restriction sites located near the promoter sequence to provide for the insertion of nucleic acid sequences encoding heterologous proteins. For example, the two multi-cloning sites provided in the pBridge vector (BD Biosciences/Clontech). A selectable marker operative in the expression host may be present. Expression vectors may be used for, among other things, the production of fusion proteins, as described above.

Expression systems may be employed, as appropriate, with prokaryotes or eukaryotes in accordance with conventional methods, depending upon the purpose for expression. For large-scale production of the protein, a unicellular organism, such as E. coli, B. subtilis, S. cerevisiae, insect cells in combination with baculovirus vectors, or cells of a higher organism such as vertebrates, e.g., COS 7 cells, HEK 293, CHO, Xenopus oocytes, etc., may be used as the expression host cells. Specific expression systems of interest include bacterial-, yeast-, insect cell-and mammalian cell-derived expression systems (see, e.g., WO03/062270; Fernandez, J. M. & Hoeffler, J. P., Gene Expression Systems—using nature for the art of expression, Academic Press 1999).

When any of the above-referenced host cells, or other appropriate host cells or organisms are used to replicate and/or express the polynucleotides or nucleic acids of the invention, the resulting replicated nucleic acid, RNA, expressed protein or polypeptide is within the scope of the invention as a product of the host cell or organism. The product may be recovered by an appropriate means known in the art.

The term “output signal” is a general term used to describe any biological event that can be detected in an assay system, such as for example, without limitation, in a transcription-based yeast two hybrid assay, a split ubiquitin assay, etc. A biologically detectable event means an event that changes a measurable property of a biological system, for example, without limitation, light absorbance at a certain wavelength, light emission after stimulation, presence/absence of a certain molecular moiety in the system, electrical resistance/capacitance etc., which event is conditional on another, possibly non-measurable or less easily measurable property of interest of the biological system, for example, without limitation, the presence or absence of an interaction between two proteins. Preferably, the change in the measurable property brought about by the biologically detectable event is large compared to natural variations in the measurable property of the system. Examples include the yellow color resultant from the action of β-galactosidase on o-nitrophenyl-b-D-galactopyranoside (ONPG) (J. H. Miller, Experiments in Molecular Genetics, 1972) triggered by transcriptional activation of the E. coli lacZ gene encoding α-galactosidase by reconstitution of a transcription factor upon binding of two proteins fused to the two functional domains of the transcription factor. Other examples of biologically detectable events are readily apparent to the person skilled in the art. Alternatively, other biological functions may be induced and detected following oligomerization, preferable dimerization, of the output-inducing domains. For example, transcriptional regulation, secondary modification, cell localization, excocytosis, cell signaling, protein degradation or inactivation, cell viability, regulated apoptosis, growth rate, cell size. Such biological events may also be controlled by a variety of direct and indirect means including particular activities associated with individual proteins such as protein kinase or phosphatase activity, reductase activity, cyclooxygenase activity, protease activity or any other enzymatic reaction dependent on subunit association. Also, one may provide for association of G proteins with a receptor protein associated with the cell cycle, e.g. cyclins and cdc kinases, or multiunit detoxifying enzymes.

In some embodiments, an output-inducing peptide comprises the TAG molecule, described in greater details below (see also Table I, infra). Examples of TAG molecules include epitope tags, affinity tags, DNA binding domains, Src polypeptide that produces a myristoylation signal, or other molecules.

In some embodiments, an output-inducing peptide comprises the Marker molecule, described in greater details below (see also Table I, infra). Examples of Marker molecule include a transcriptional activation domain, hSos polypeptide, affinity tags, epitope tags, or other molecules.

In preferred embodiments, an output-inducing peptide in conjunction with a conventional or modified two-hybrid or interaction trap system, described in greater detail below, may comprise the DNA binding domain (“DBD”) of a transcription factor, e.g., an activator. Alternatively, an output-inducing peptide comprises the activation domain (“AD”) of a transcription activator. In a preferred embodiment, a Ub or U1 nucleic acid coding sequence is fused to the nucleic acid sequence of a DBD, e.g., DBD of GAL4, a transcription activator for the β-galatosidase gene, to create the bait fusion peptide encoding sequence, and a prey fusion peptide encoding sequence comprises an AD, e.g. AD of GAL4. In this embodiment, the detectable signal comprises expression of a reporter gene operably linked to a transcriptional regulatory sequence or element that is responsive to the transcription activator from which the DBD and the AD (or at least the DBD itself) are derived. As used herein, the term “reporter gene” refers to a coding sequence attached to heterologous promoter or enhancer elements and whose product may be assayed easily and quantifiably when the construct is introduced into tissues or cells.

Also provided are other DBDs and ADs that can be used in a yeast or mammalian two-hybrid system, as described below.

Also provided are nucleic acids that encode fusion proteins of Ub or Ub1 or a prey peptide of the present invention, or fragments thereof, fused to a second peptide or protein. The second protein may be, for example, a degradation sequence, a signal peptide, or any protein of interest.

As will be understood by skilled artisans, a Ub or Ub1 of the present invention may be used without being fused to another protein. Detailed description is provided below, where different methods and systems used in the art to detect protein-protein interaction or the formation of a protein complex are described. Specifically, in a phage display system, a Ub or Ub1 can be immobilized by ways other than a linker/anchoring peptide.

Similarly, candidate protein substrates for Ub or Ub1-conjugation machinery of the present invention are not necessarily fused to another protein.

In other embodiments, an output-inducing peptide comprises a fluorescent protein, and the detectable signal is a change in fluorescence.

Preferably, the output-inducing peptide fused to Ub or Ub1 produces a fluorescent signal distinct from the fluorescent signal, e.g., different colors, produced by the output-inducing peptide fused to a prey peptide. Thus, combined fluorescent signals due to the physical proximity of the two fusion proteins will exhibit a change in fluorescence, in comparison to two distinct fluorescent signals, that is detectable by technologies available in the art, e.g., fluorescence microscopy.

The use of fluorescent proteins derived from Aequorea victoria has revolutionized research into many cellular and molecular-biological processes. In a preferred embodiment, the output-inducing peptide comprises a fluorescent protein. The gene sequence encoding a fluorescent protein may be joined in-frame with a gene encoding the protein of interest, e.g., a Ub or Ub1 or a prey peptide, and the desired fusion protein produced when inserted into an appropriate expression vector. Possible expression vectors are described above and specific examples for two-hybrid or interaction trap systems are provided below. For example, polymerase chain reaction or complementary oligonucleotides may be employed to engineer a polynucleotide sequence corresponding to the fluorescent protein, 5′ or 3′ to the gene sequence corresponding to the protein of interest. Alternatively, the same techniques may be used to engineer a polynucleotide sequence corresponding to the fluorescent protein sequence 5′ or 3′ to the multiple cloning site of an expression vector prior to insertion of a gene sequence encoding the protein of interest. The polynucleotide sequence corresponding to the fluorescent protein sequence may comprise additional nucleotide sequences to include cloning sites, linkers, transcription and translation initiation and/or termination signals, labelling and purification tags.

Several examples of fluorescent proteins are known in the art. A well-known example of a fluorescent protein is the native GFP derived from species of the genus Aequorea, suitably Aequorea victoria. The chromophore in wtGFP (native GFP) from Aequorea victoria is at positions 65-67 of the predicted primary amino acid sequence.

The bait and/or prey fusion proteins of the present invention may comprise a wtGFP or a fragment thereof that can generate a detectable fluorescence signal.

U.S. Pat. No. 5,491,084 describes the use of GFP as a biological reporter. Early applications of GFP as a biological reporter (Chalfie et al. Science, (1994), 263, 802-5; Chalfie, et al, Photochem. Photobiol., (1995), 62 (4), 651-6) used wild type (native) GFP (wtGFP), but these studies quickly demonstrated two areas of deficiency of wtGFP as a reporter for use in mammalian cells. Consequently, significant effort has been expended to produce variant mutated forms of GFP with properties more suitable for use as an intracellular reporter.

A number of mutated forms of GFP with altered spectral properties have been described. A variant-GFP (Heim et al. (1994) Proc. Natl. Acad. Sci. 91, 12501) contains a Y66H mutation which blue-shifts the excitation and emission spectrum of the protein. W096/27675 describes two variant GFPs, obtained by random mutagenesis and subsequent selection for brightness, which contain the mutations V163A and V163A+S175G, respectively. These variants were shown to produce more efficient expression in plant cells relative to wtGFP and to increase the thermo-tolerance of protein folding. The double mutant V163A+S175G was observed to be brighter than the variant containing the single V 163A mutant alone. This mutant exhibits a blue-shifted excitation peak. U.S. Pat. No. 6,172,188 describes variant GFPs wherein the amino acid in position 1 preceding the chromophore has been mutated to provide an increase of fluorescence intensity. Such mutations include F641, F64V, F64A, F64G and F64L, with F64L being the preferred mutation. These mutants result in a substantial increase in the intensity of fluorescence of GFP without shifting the excitation and emission maxima. F64L-GFP has been shown to yield an approximate 6-fold increase in fluorescence at 37° C. due to shorter chromophore maturation time.

In addition to the single mutants or randomly derived combinations of mutations described above, a variety of variant-GFPs have been created which contain two or more mutations deliberately selected from those described above and other mutations, and which seek to combine the advantageous properties of the individual mutations to produce a protein with expression and spectral properties which are suited to use as a sensitive biological reporter in mammalian cells. U.S. Pat. No. 6,194,548 discloses GFPs with improved fluorescence and folding characteristics at 37° C. that contain, at least, the changes F64L and V163A and S175G.

U.S. Pat. No. 5,777,079 describes a blue fluorescent protein (BFP) containing F64L, S65T, Y66H and Y145F mutations. This is referred to as BFP, because it emits blue fluorescence by UV excitation (R. Heim et al. Curr. Biol. (1996), 6,178-182; R. Heim et al. Proc. Natl. Acad. Sci. USA, (1994), 91,12501-12504). However, this BFP was very dim and it experienced severe photo-bleaching as compared to green fluorescent protein. U.S. Pat. No. 6,194,548 describes a further BFP containing the F64L, Y66H, Y145F and L236R substitutions. This patent also discloses a mutant containing: F64L, Y66H, Y145F, V163A, S175G, and L236R. Further mutants are described comprising the Y66H, Y145F, V163A and S175G mutations; and the F64L, Y66H, and Y145F mutations. Further optional mutations are described at S65T and Y231 L. These mutants are more photostable than those described in U.S. Pat. No. 5,777,079.

WO03029286 describes novel engineered derivatives of blue fluorescent protein (BFP) and nucleic acids that encode engineered BFPs which exhibit more stable fluorescence properties and have different excitation spectra and/or emission spectra relative to wtGFP when expressed in non-homologous cells at temperatures above 30° C., and when excited at about 390 nm. In particular, WO03029286 provides novel fluorescent proteins that fluoresce in the blue region of the spectrum (“BFPs”) and have a cellular fluorescence that is more stable than that of BFPs previously described.

WO03062270 describes a colorless protein, acGFP, from Aequorea coerulescens, or fluorescent and non-fluorescent mutants or derivatives of acGFP, as well as fragments and homologs of the nucleic acid compositions. The phrase “fluorescent protein” means a protein that is fluorescent, e.g., it may exhibit low, medium or intense fluorescence upon irradiation with light of the appropriate excitation wavelength. The proteins disclosed in WO03062270 are those in which the fluorescent characteristic is one that arises from the interaction of two or more amino acid residues of the protein, and not from a single amino acid residue. As such, the fluorescent proteins of WO03062270 do not include proteins that exhibit fluorescence only from residues that act by themselves as intrinsic fluors, i.e., tryptophan, tyrosine and phenylalanine. Instead, the fluorescent proteins of WO03062270 are fluorescent proteins whose fluorescence arises from some structure in the protein other than the above-specified single amino acid resides; e.g., it arises from an interaction of two or more amino acid residues.

Accordingly, fusion proteins of the present invention may comprise a BFP, selected from the variants described above.

Fusion proteins of the present invention may comprise for example, an acGFP or mutant acGFP polypeptide, as described in WO03/062270 and a second polypeptide (a Ub or Ub1 or a prey peptide) fused in-frame at the N-terminus and/or C-terminus of the acGFP polypeptide.

In a preferred embodiment, the bait fusion polypeptide of the present invention comprises a fluorescent protein that is distinct from the fluorescent protein as part of the prey fusion polypeptide. For example, the bait fusion polypeptide comprises a GFP, and prey fusion polypeptide comprises a BFP. When these two fusion polypeptides are brought in physical proximity with each other, an output signal of the present invention comprises the change in the fluorescence from a single color to a combination of green and blue.

The present invention is based on the Ub or Ub1-conjugation or ubiquitination machinery in which E3 may function as a substrate recognition protein or a ligase. In the presence of the ubiquitination machinery, Ub or Ub1 will be conjugated onto its protein substrate and thereby form a protein complex. Accordingly, any method or system capable of detecting protein-protein interaction or the formation of a protein complex may be modified, to reflect the dependency on the presence of a ubiquitination machinery, to practice the present invention. Examples of methods and systems are listed in Table I.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature. An interaction can be direct or indirect, i.e., mediated by another molecule. It is noted that ubiquitination or similar protein modification also represents a form of protein-protein interaction in the present invention, that is, a ubiquitinated substrate protein is basically a protein complex of ubiquitin and the substrate protein formed by protein-protein interaction by a covalent link. It is also noted that the protein-protein interaction by a covalent link is mediated or catalyzed by a ubiquitination machinery.

Methods for identifying protein substrates for ubiquitination or similar protein modifications include yeast and mammalian two hybrid-type assays, as well as phage display-type methods and the presence or absence (which can be used as a control to select ubiquitinated substrate proteins instead of other Ub- or Ub1-interacting proteins) of a ubiquitination machinery in these assays. These methods present the advantage that the nucleic acid encoding the interacting peptides is simultaneously identified, as opposed to other methods. These methods also have the advantage that make it feasible to conduct high throughput screening for the desired protein substrates.

TABLE I

| | | Marker molecule- | |
|---|---|---|---|
| | TAG molecule- [Ub or | [candidate interaction | |
| METHOD | Ub1] | domain] | Reference |
| 1. Yeast Two-Hybrid | GAL 4 or lex A | B42, VP16, or GAL4 | (Gyuris et al. (1993) |
| or Interaction Trap | Polypeptide | Polypeptide | Cell 75: 791-803) |
| | [DNA Binding | [Transcriptional | & (Fields et al. |
| | Domains] | Activation Domain] | (1994) Trends in |
| | | | Gen. 10: 286-92) |
| 2. Yeast Cytoplasmic | Src Polypeptide | hSos Polypeptide | (Aronheim et al. |
| Two-Hybrid | [myristoylation signal] | [GEF, mammalian guanyl | (1997) Mol Cell. |
| [SRS, SOS | | nucleotide exchange | Biol. 17: 3094-3102) |
| Recruitment System] | | factor] | |
| 3. Mammalian | GAL 4 or lex A | VP16 Polypeptide | (Luo et al. (1997) |
| Two-Hybrid | Polypeptide | [herpes simplex virus | BioTechniq. |
| or Interaction Trap | [DNA Binding | transcriptional activator] | 22: 350-2) |
| | Domains] | | |
| 4. Far-Western | Radioactive atoms, | None or | (see e.g. Bonardi et |
| [related to Western | Epitope Tags, Affinity | Expression Vector Fusion | al. (1995) Bioch. |
| except detection is by | Tags | Polypeptide | Biophys. Res. |
| interaction with a | [e.g. ³⁵S-met, ³²P, or | | Comm. 206: 260-5) |
| protein other than an | ¹²⁵I; HA or FLAG; or | | |
| antibody] | biotin or polyHis] | | |
| 5. Phage Display | Affinity Tags | bacteriophage coat | (see e.g. Smith |
| | [e.g. biotin, polyHIS to | protein | (1985) Science 228: |
| | facilitate | [e.g. filamentous phage | 1315-17 & Johnson |
| | immobilization to a | gIII or gVIII coat proten] | et al (1993) Curr. |
| | sold support matrix] | | Opin. Struc.Bio. 3: |
| | | | 564) |
| 6. Protein Trap + Nucleic | Affinity Tags | Lac repressor proten | (U.S. Pat. Nos. |
| Acid Snag | [e.g. biotin, polyHIS to | [lacl; with lac operator | 5,270,170; |
| [Affymax Peptide | facilitate | incorporated into the | 5,338,665; & |
| Library | immobilization | expression vector] | 5,498,530) |
| & screening Method] | to a solid support | | |
| | matrix] | | |
| 7. Biomolecular | Affinity Tags | None, Affinity Tag | (see e.g. Fivash et |
| Interaction Analysis | [e.g. polyHIS to | [isolated proteins may be | al. |
| [e.g. Pharmacia | directly | used w/o prior | (1998) Curr. Opin. |
| BIAcore | link to a Ni-based chip | modification; method of | Biotechnol. 9: 97-101 |
| surface plasmon | or | producing protein may, in | & Schuck |
| resonance detection] | a DNA binding domain | some instances, introduce | (1997) Annu. Re. |
| | to link to a detection | an affinity purification | Biophys. Biomol. |
| | chip | tag] | Struct. |
| | via a DNA oligo] | | 26: 541-66) |
| 8. Peptide Matrix | None; or modified to | Solid Support Matrix | (e.g. U.S. Pat. |
| Arrays | Support detection | [in situ synthesis of | Nos. 5,653,949; |
| [e.g. Affymax | [e.g. fluorescently | random polypeptides an | 5,679,773; |
| combinatorial peptide | tagged] | associated identification | 5,690,894; |
| matrix arrays] | | tag address] | 5,708,153; & |
| | | | 5,744,305) |

In a preferred method, a protein substrate for an E3-mediated ubiquitination is obtained using the yeast “two-hybrid” or “interaction trap”-like method. The yeast two-hybrid or interaction trap assay has been developed as a means of detecting specific protein-protein interactions thereby allowing for the assessment of such interactions between known components of a biochemical pathway or macromolecular assemblage as well as allowing for the cloning of novel components of such pathways and assemblages. One aspect of the present invention pertains to the use of a ubiquitination machinery and Ub or Ub1 to clone other protein substrates that can be modified by the Ub or Ub1. In a preferred embodiment of the invention, mammalian Ub or Ub1 is used as the “bait” in a two hybrid or interaction trap cloning procedure.

Briefly, the conventional yeast two-hybrid assay relies upon the detection of a transcriptional activation signal delivered to a reporter gene. This transcriptional activation signal is generated by the reconstitution of a reporter gene-specific transcriptional activator from covalently separate DNA binding and transcriptional activation domains via a specific protein-protein interaction.

Two-hybrid or interaction trap systems are generally based on the finding that most eukaryotic transcription activators are modular and can be divided into two distinct domains, a DBD and a transcriptional AD. Furthermore, it has been shown that the DBD does not have to be covalently linked to the AD, so long as the two separate polypeptides interact, or come in physical proximity with one another, for the complex to function as a transcriptional activator (Maetal.(1988) Cell 55: 443-446). The interaction trap system relies upon this fact to clone proteins which interact with one another by virtue of their ability to noncovalently reconstitute a transcriptional activator when they are independently fused to the two distinct domains of a “third party” synthetic transcriptional activator. In particular, the method makes use of two chimeric genes, for example, a bait fusion peptide comprising a Ub or Ub1 and a DBD and a prey fusion peptide comprising a candidate protein substrate and an AD, which independently express a DBD hybrid or fusion protein and a transcriptional AD hybrid or fusion protein.

In some embodiments, a bait fusion peptide comprises the coding sequence for a DNA-binding protein, such as the bacterial repressor protein LexA, fused in frame to the coding sequence for a Ub or Ub1. The prey fusion peptide comprises the coding sequence for a transcriptional AD, such as the transcriptional activator sequence B42 (Ma and Ptashne (1987) Cell 51: 113-119), fused in frame to a gene from a cDNA library which encodes a selection of polypeptides representing various candidate protein substrates for a tested ubiquitination machinery. The bait fusion peptide can also be thought of as a specific form of the “Ub or Ub1 trap” (i.e., Ub- or Ub1-TAG, wherein the TAG entity is the LexA DBD in certain embodiments). Both the bait and prey fusion peptides are expressed simultaneously in a yeast cell. If the bait and prey fusion proteins are able to interact, e.g., form a protein complex, they bring into close proximity the LexA DBD and the B42 synthetic AD, thereby reconstituting a transcriptional activator protein with the DNA recognition specificity of LexA.

In preferred embodiments, the bait and prey fusion proteins interact in the presence of a ubiquitination machinery, and the prey fusion protein is ubiquitinated or modified by the Ub or Ub1 from the bait fusion peptide. It is conceivable that more interaction prey fusion peptides will be detected and/or identified when a ubiquitination machinery is present in the assays as compared to the absence of the machinery, due to the fact that candidate protein substrates only interact with Ub or Ub1 (i.e., become ubiquitinated or modified) when the machinery is present to catalyze the ubiquitination or modification.

It is further noted that different protein substrates can be identified using different ubiquitination machinerys comprising different E3s and/or E2s. It is an object of the present invention to utilize different E3s, which function as auxiliary substrate recognition proteins in a ubiquitination machinery, to identify different protein substrates subject to ubiquitination or similar protein modification.

Preferably, a fusion protein, e.g., a bait fusion peptide, of present invention comprising a Ub or Ub1 is a N-terminal fusion protein of Ub or Ub1, that is, the C-terminus of the Ub or Ub1 is free.

A third hybrid gene contained in the same cell may be used to detect the presence of this “noncovalently” reconstituted transcriptional activator. The third hybrid gene may comprise a reporter gene which is operably linked to a DNA sequence comprising a binding site for the DBD of the first hybrid gene, in certain embodiments the LexA operator. The “noncovalently” formed transcriptional activator (in this case LexA//B42) recognizes this lexA DNA binding sequence operably linked to the reporter and causes the expression of this third hybrid reporter gene which can be detected and used to score for the interaction of the bait and prey fusion proteins. Again, the presence or absence of a ubiquitination machinery in the same cell may indicate the presence of desired protein substrates for Ub or Ub1 used herein.

In a preferred embodiment of the invention, two or more reporter genes, each operably linked to the same DNA binding recognition sequence, are present in the same yeast cell in the presence of the bait and prey fusion peptides. As an example, one of the reporters could encode an easily assayed heterologous enzyme activity, such as the bacterial LacZ gene which encodes a β-galactosidase enzyme activity capable of being detected and measured using a chromogenic substrate such as X-gal which is converted to a blue chromophore in the presence of β-galactosidase enzyme activity. Further, the same cell could contain a second reporter gene comprising the coding sequence for the yeast LEU2 gene. The same haploid yeast cell would also preferably contain a deleted or otherwise mutant allele of the naturally occurring chromosomal copy of the LEU2 gene, thereby making growth on leucine-deficient media solely dependent upon expression of the LEU2 hybrid reporter gene. If a bait fusion protein, LexA-Ub or Ub1 for example, is conjugated by a ubiquitination machinery onto the product of a prey fusion protein, the synthetic activator B42 fused to a candidate protein substrate, then the resulting reconstituted third party transcriptional activator LexA//B42, would bind to and activate both of the third hybrid gene reporters resulting in both the complementation of this yeast strain's leucine auxotrophic phenotype, due to activation of the LEU2 reporter, and blue colony color on X-gal containing medium, due to activation of the LacZ reporter.

In another embodiment of this two hybrid or interaction trap screening assay, one of the two third hybrid gene reporters is the yeast HIS3 gene and the haploid yeast cell also contains a deleted or otherwise mutant allele of the naturally occurring chromosomal copy of the HIS3 gene, thereby making growth on histidine-deficient medium solely dependent upon expression of the HIS3 hybrid reporter gene. In this instance, the protocol can be adapted for use either with bait fusion proteins which otherwise independently (i.e., cryptically) weakly activate transcription on their own in the absence of the prey fusion proteins or in the specific identification and isolation of proteins that interact with the bait fusion protein to such a degree that the resulting expression of the third hybrid gene reporters is of a sufficient strength so as to surpass a predetermined threshold. These applications are made possible by the addition of Aminotriazole (3-amino-1,2,4-triazole or 3-ATZ) to the media used in the screen. 3-Aminotriazole is a competitive inhibitor of the histidine anabolic enzyme activity encoded by the Saccharomyces cerevisiae HIS3 gene product (see, e.g., Erickson and Hannig (1995) Yeast I 1: 157-67). The addition of 3-ATZ to media lacking histidine results in a condition where the abovementioned yeast strain must evince sufficiently strong interaction between the first and second hybrid gene products so as to create a sufficiently high steady state level of the reconstituted third party transcriptional activator, thereby stimulating HIS3 reporter expression sufficiently so as to overcome the competitive inhibition of the HIS3 gene product by 6-ATZ.

As stated above, this technique is also adaptable to instances wherein the abovementioned Ub or Ub1-TAG (or bait fusion protein) is sufficient to cause activation of the third hybrid gene reporters on its own, in the absence of a prey fusion protein. In this instance where the “bait” is found to cryptically activate transcription on its own, or is known to function naturally as a transcriptional activator, 3-ATZ can be added to suitable media lacking histidine until the appropriate level of 3-ATZ sufficient to block the level of activity of the product of the HIS3 reporter expressed in the presence of the first hybrid gene alone. This level of 3-ATZ can then be added to the media on which the two hybrid screen is performed so that complementation for growth on histidine deficient media now depends upon the higher levels of expression of the HIS 3 reporter obtained when the product of the first hybrid gene interacts with the product of the second hybrid gene as compared to the level of expression obtained from the HIS 3 reporter in the presence of the first hybrid gene alone.

In other embodiments of this two hybrid or interaction trap method, any of a number of the elements of the system can be modified. For example, Fields and his coworkers (see e.g. U.S. Pat. No. 5,667,973) devised one version of the interaction trap in which the DNA binding entity of the first hybrid gene product is the DBD of the yeast transcriptional activator GAL4, but can otherwise be the DBD of any transcriptional activator having separate DBDs and transcriptional ADs such as those of the yeast GCN4 and ADRI proteins, and the transcriptional AD of the second hybrid gene product is the GAL4 transcriptional AD. In this case, a yeast strain which is null or deficient for its normal chromosomal copy of the GAL4 gene and which contains bait and prey fusion proteins that interact, can be selected for directly on media containing galactose as the sole carbon source because the reconstituted third party transcriptional activator (GAL4 DNA BIND//GAL4 TSX ACT) will drive expression of the necessary galactose catabolic enzyme activities including the products of the GAL1 and GAL10 genes. If the same yeast strain also contains a GAL1-lacZ third hybrid gene reporter, then these same transformants can also be screened—for blue color on X-gal/galactose media where the intensity of blue color detected will be directly related to the strength of the interaction between the bait and prey fusion proteins. In some instances, it may be preferable that the yeast contain another hybrid gene, such as GAL1-HIS3, in which the GAL1 transcriptional regulatory sequences are fused to the structural gene of HIS3. This third hybrid gene allows for direct selection of prey fusion proteins comprising candidate protein substrates by growing the yeast strain (further comprising a ubiquitination machinery) on galactose media in the absence of exogenous histidine. In this particular example, the use of 3-ATZ in the growth media, as described above, can be applied in situations where the bait fusion protein alone serves as a weak transcriptional activator.

In preferred embodiments of the present invention, the interaction between a candidate protein substrate fusion peptide (or prey fusion peptide) and a Ub- or Ub1-TAG trap (or bait fusion peptide) will be further determined for its dependency upon the presence of a ubiquitination machinery. The ubiquitination machinery preferably comprises an E3 of choice (i.e., of which protein substrates are of particular interest, POSH for example) which would be exogenous to the yeast or mammalian cell used in the interaction trap assay (i.e., the host cell). The ubiquitination machinery may further comprises exogenous E1s and/or E2s or depend on the host cell's components of a ubiquitination machinery.

The method of the present invention allows for the use of any of a number of different reporter genes whose expression is driven by the physical association of the bait and prey fusion proteins in the presence of a ubiquitination machinery. The choice of reporter gene will depend upon the particular circumstances such as the ease of selection or assay of such genes. Such genes include, without limitation, lacZ, amino acid biosynthetic genes (e.g. the yeast LEU2, HIS3, TRP I, or URA3 genes, nucleic acid biosynthetic genes, the mammalian chloramphenicol acetyltransferase (CAT) gene or GUS gene, or any surface antigen gene for which specific antibodies are available. Reporter genes may encode any enzyme that provides a phenotypic marker, for example, a protein that is necessary for cell growth or a toxic protein leading to cell death, or one encoding a protein detectable by color assay or one whose expression leads to an absence of color. Particularly preferred reporter genes are those encoding fluorescent markers, such as the GFP gene and variants thereof. Reporter genes may facilitate either a selection or a screen for reporter gene expression, and quantitative differences in reporter gene expression may be measured as an indication of interaction affinities.

It is understood that the method of the present invention allows for the use of any of a number of DBDs in the construction of the first hybrid gene (i.e., the bait fusion peptide coding sequence). Thus, in addition to 1 exA and GAL4 DBDs, other DBDs that are well known in the art include the DBDs of the proteins ACE 1, CUP 1, lambda cI, lac repressor, Jun, Fos, or GCN4. The method provides for the use of these alternative DBDs by way of additionally altering the third hybrid or reporter gene construct (or constructs) such that it contains a fragment of DNA encompassing the binding site of the alternative DBD, and wherein said binding site is operably linked to the reporter gene(s). These DBDs are considered as possible TAGs, as shown in Table I.

The Marker Molecule of a test polypeptide, e.g., a candidate protein substrate, (see Table I) is meant to facilitate identification of a candidate protein substrate of interest and isolation of its encoding nucleic acid. In the yeast two-hybrid embodiments of the present invention, the Marker is typically a transcriptional AD which functions in yeast and which is a component of the second hybrid gene (i.e., the prey fusion peptide coding sequence). It is understood that the second hybrid gene of the present invention can encode any of a number of alternative transcriptional ADs including the GAL4 transcriptional activation domain region 11, the strong transcriptional activator VP 16, the weak synthetic transcriptional activators B17 and B112, or the amphipathic helix domain described in Giniger and Ptashne ((1987) Nature 330: 670). Modifications of the transcriptional activation can be particularly useful when attempting to either increase or decrease the sensitivity of the screening assays. In the method of the present invention the prey fusion protein may further contain, in addition to a transcriptional AD, an optional nuclear localization sequence, such as that of the SV40 Large T antigen encoded by the amino acid sequence PPKKKRKVA, which allows for the requisite partitioning of the prey fusion protein in cases where the prey moiety is normally exclusively cytoplasmic. The prey fusion protein may additionally contain an epitope tag, such as hemagluttinin or FLAG, so that production of full length prey fusion proteins can be confirmed in a Western blot.

Epitope tags may further provide a convenient means of testing for covalent linkage of the bait and prey moieties as is anticipated in preferred embodiments of this invention. This determination is conveniently made by means of a Western blot analysis and provides a biochemical means of classifying the clones obtained from a Ub- or Ub1-trap screen.

It is further understood that in the method of the present invention the nature of the cDNAs used in constructing nucleic acids encoding the prey fusion proteins can be tailored to various applications of the Ub- or Ub1-trap cloning method. In particular, cDNAs may be constructed from any mRNA population and inserted into an equivalent vector for the expression of the prey fusion protein. Such a library of choice may be constructed de novo using commercially available kits (e.g., from Stratagene, LaJolla, Calif.) or using well established preparative methods. Alternatively, a number of cDNA libraries (from a number of different organisms) are publicly and commercially available; sources of libraries include, e.g. BD Biosciences/Clontech and Stratagene (La Jolla, Calif.) as well as publicly available libraries such as those described and summarized on the internet (see www.fccc.edu/research/labs/golemis/IT-libraries.html).

It is worth noting that many commercially available yeast two-hybrid systems have been created, many of which have particular advantages and all of which are understood to be adaptable to, and therefore aspects of, the present invention. For example, the Invitrogen (Carlsbad, Calif.) Hybrid Hunter™ System makes use of the drug Zeocin and a drug resistance marker (ZeOR) to maintain selection for the bait fusion protein-encoding vector. This modification of the method allows greater compatibility with other yeast two-hybrid libraries (i.e., prey fusion protein-encoding vector systems) as well freeing a useful selectable prototrophy marker for use in modifications of the standard two-hybrid protocol. Such modifications of the standard two-hybrid protocol may involve, for example, the introduction of a library of test polypeptides to identify proteins capable of potentiating ubiquitination or similar protein modification of substrate proteins that would not normally be ubiquitinated or modified (see, e.g., the “three hybrid” system described in SenGupta et al. (1996) Proc. Natl. Acad. Sci. USA 93: 8496-8501). This modification of the system may be useful in identifying polypeptide agonists of the ubiquitination or similar protein modification machinery. Alternatively, a library of test polypeptides may be introduced into a yeast strain already expressing a bait and prey fusion protein interaction pair (e.g., Ub or Ub1 and an identified protein substrate in the presence of a ubiquitination machinery) and polypeptides capable of disrupting this interaction may be selected (see, e.g., the “split hybrid” system described in Shih et al. (1996) Proc. Natl. Acad. Sci. USA 93: 13896-901). This modification of the system may be useful in identifying polypeptide antagonists of the ubiquitination or similar protein modification machinery.

It is further noted that the candidate proteins that are part of the prey fusion proteins do not need to be naturally occurring full-length polypeptides. For example, a candidate protein may be encoded by a “domain” library of small partial cDNA sequences which can be obtained by internal primmer of cDNA synthesis with random (non-polyT) primers and selection of appropriate sized partial cDNA fragments (e.g. <1 kb). Alternatively the candidate protein entity of the prey fusion protein may correspond to a synthetic sequence or may be the product of a randomly generated open reading frame or a portion thereof. This particular embodiment is also usefully employed in the development of therapeutics which modulate the activity of the Ub- or Ub1-trap moiety. This particular embodiment of the Ub- or Ub1-trap method, in which a purely synthetic ubiquitination protein substrate is sought, is also readily adapted to the phage display and peptide matrix display embodiments of the present invention.

In still other applications of the two-hybrid system, the bait and prey fusion proteins are independently expressed in haploid yeast strains of opposite mating type. For example, a single homogeneous strain expressing a Ub- or Ub1-TAG/DBD can be established by transforming a yeast strain having the appropriate two-hybrid driven (e.g. GAL4ₒₚor lexAₒₚ-driven) third hybrid gene selectable markers and/or reporters with the bait fusion protein-encoding vector. A heterogeneous population of prey fusion protein expressing yeast cells is then created by means of high efficiency transformation of a second haploid yeast strain of opposite mating type. This heterogeneous population is then mated to the yeast strain of opposite mating carrying the first hybrid gene. Candidate protein substrates represented by the prey fusion protein population can be selected for by requiring expression of the third hybrid gene selectable marker. This can be achieved by plating on the appropriate selective media. This “mass mating” protocol obviates repetition of the most difficult step (i.e., high-efficiency transformation of a yeast strain with a prey fusion protein-encoding “prey” library) when performing repeated screen with different bait fusion proteins.

Since other eukaryotic cells use a mechanism similar to that of yeast for transcription, such cells, including mammalian cells such as HeLa, can be used instead of yeast to test for protein-protein interactions with the Ub- or Ub1-fusion protein (a bait fusion protein). In particular, the method of the present invention can be employed in a mammalian two-hybrid assay (Luo et al., (1997) Biotechniques 22: 350-352). In this adaptation of the yeast two-hybrid system, the bait and prey fusion proteins are expressed from mammalian promoters in a mammalian cell. As in the yeast nuclear two-hybrid method described above, the Ub- or Ub1-TAG is encoded by a first hybrid gene, in which the TAG moiety comprises a polypeptide DBD, and a library of test polypeptides is expressed by a population of prey fusion protein-encoding vectors comprising a collection of cDNA sequences fused to a polypeptide transcriptional AD. In one example of a preferred embodiment, interaction of the candidate protein substrate tagged with a VP16 transcriptional AD with a Ub or Ub1 fused to a GAL4 DBD drives expression of reporters that direct the synthesis of Hygromycin B phosphotransferase, Chloramphenicol acetyltransferase, or CD4 cell surface antigen (Fearon et al. (I 992) PNAS 89: 7958-62). In another, interaction of these bait and prey fusion proteins drives the synthesis of SV40 T antigen, which in turn promotes the replication of the prey plasmid, which carries an SV40 origin (Vasavada et al. (1991) PNAS 88: 10686-90). Suitable promoters for expression of the bait and prey fusion proteins in mammalian cells include strong viral promoters such as those from CMV and SV40 or weaker cellular promoters such as that from the tk (thymidine kinase) gene. The vectors that express these fusion proteins are cotransfected with a third hybrid gene encoding a reporter such as chloramphenicol acetyltransferase (CAT) or beta-galactosidase into a mammalian cell line. The reporter of the third hybrid gene, which contains upstream DNA binding sites specific for the DBD of the first hybrid gene, can alternatively be integrated into the mammalian genome by prior transfection, selection, and clonal isolation and characterization. If the two fusion proteins (bait and prey) interact, there will be a significant increase in the expression of the reporter gene which can be detected and assayed using the appropriate reagents. As described above, the interaction between the two fusion proteins in preferred embodiments will be determined for its dependency on the presence of a ubiquitination machinery.

This mammalian screening technique, by using small tissue culture samples, can be adapted for use in high throughput screens. The mammalian two-hybrid has two main advantages: assay results can be obtained within 48 hours of transfection and protein interactions in mammalian cells may better mimic actual in vivo interactions, particularly in the case where the relevant interaction is dependent upon mammalian post-translational modifications (including phosphorylations and glycosylations) of the bait and/or prey fusion proteins or in instances where the bait and prey fusion proteins interact indirectly through the action of a third molecule (such as a protein) which is endogenous to mammalian cells but not to yeast cells. In the latter instances, the third molecule likely acts upon the ubiquitination machinery which is responsible for the interaction between the bait and prey fusion proteins in preferred embodiment.

As described above, the present invention further determines whether any interaction detected between a bait fusion protein (e.g., Ub- or Ub1-TAG, TAG can be a DBD) and prey fusion protein with an AD is dependent on the presence of a ubiquitination machinery of choice. Such machinery may comprise an exogenous E3, introduced to the mammalian host cell by, e.g., an expression vector. Such machinery may utilize the mammalian host cell's own E1s and/or E2s to complete the machinery of choice, or utilize all exogenous components to complete the machinery of choice.

The conventional two-hybrid system, as described above, is based on a transcriptional readout and may not be suitable for either identifying transcriptional repressors that are protein substrates for a ubiquitination or similar protein modification machinery of the present invention. A transcriptional repressor may, for example, prevent transcriptional activation resulting from recruitment of the transcriptional AD in the prey fusion protein. A novel screen for detecting protein-protein interactions that is not based directly on the formation of a hybrid transcriptional activator has been developed by Michael Karin and his colleagues and termed the SRS or SOS recruitment system (Aronheim et al. (1997) Mol. Cell. Biol. 17: 3094-3102). As summarized in Table I, this embodiment of the Ub- or Ub1-trap, the polypeptide TAG in the bait fusion protein is typically a Src derived polypeptide myristoylation signal, which when expressed in vivo is joined to a membrane lipid and directed to the cell membrane; while the molecular marker in the prey fusion protein is typically a guanyl nucleotide exchange factor such as mammalian hSos. This cytoplasmic two-hybrid assay system involves the use of a defective ras/raf cytoplasmic signaling pathway in yeast. (White, et al. (1995) Cell 80: 433-541). In this system, the mammalian guanyl nucleotide exchange factor (GEF) hSos is recruited to the plasma membrane in a Saccharomyces cerevisiae strain harboring a temperature-sensitive Ras GEF. At nonpermissive temperatures, the Cdc25-2 allele of Ras GEF is inactive and thus growth becomes dependent on the ability of a heterologous protein/protein interaction to facilitate recruitment of hSos to the plasma membrane, resulting in the stimulation of the Ras-dependent signaling cascade. The two fusion proteins necessary to utilize this system are analogous to the bait and prey fusion proteins of the yeast two-hybrid method described above, except a membrane localization signal, as opposed to a transcriptional activation signal, is reconstituted from these two components as detailed below. In the SRS system the bait fusion protein corresponds to a DNA encoding a myristoylation signal, such as that from Src, which is fused in frame to the coding sequence of a Ub or Ub1, and accordingly, the bait fusion protein comprises a Src mynistoylation signal-Ub or Ub1 fusion.

The prey fusion protein of the SRS technique is comprised of the coding sequence for hSos fused in-frame to the coding sequence of a sample gene from a cDNA library.

Because the interaction of bait and prey is assayed by reconstitution of a cytoplasmic signal transduction pathway necessary for growth and not a nuclear transcriptional activation activity, this embodiment is particularly well suited to the situations where ubiquitination of certain protein substrates lead to transcriptional activation or transcriptional repression activities which interfere with a conventional two-hybrid assay readout. This technique also has the advantage of avoiding problems-occurring with the prey fusion proteins which otherwise independently cause activation of third hybrid gene reporters. In particular, although the problem of “cryptic” activation by the bait is avoidable when using HIS3 as a reporter in the presence of aminotriazole as described above, there is otherwise no way of completely avoiding a nonspecific background of positive prey clones of a type which further test nonspecific for the original bait. Detection of an interaction of such a prey clone with the bait fusion protein using the cytoplasmic two hybrid detection method avoids “false negatives” from this class of proteins which appears to nonspecifically activate reporter genes when allowed to localize to the nucleus.

There exist a number of techniques, known in the art, for cloning genes from conventional lambda cDNA expression libraries (such as lambda gt 11) by virtue of the ability of their encoded gene product to interact with a protein or proteins of interest. These assays are essentially modifications of a traditional “Western” protocol (see, e.g., Sambrook et al. Molecular Cloning: A Laboratory Manual CSH Press) except a non-antibody protein is substituted for the antibody used in the detection phase of the assay. It is understood that the method of the present invention includes such screening and cloning techniques as applied to screens employing a Ub- or Ub1-trap target probe (an E3 target probe is also contemplated). As summarized in Table I, such “Far-Western” embodiments of the Ub- or Ub1-trap method employ radioactive atoms, epitope tags or affinity tags to serve as target polypeptide “TAG” entities, and require no specific molecular marker moiety to mark the candidate protein substrate peptides. In many cases, however, the candidate protein substrate polypeptides are from phage cloning vectors (e.g. lambda gt11) as fusions to expression vector polypeptide-encoding sequences such as LacZ or LacZ fragments. Traditional “Far-Western” screening techniques typically employ phage lambda cDNA libraries produced from various sources of cellular mRNA, depending upon the application. These libraries are then plated at a low m.o.i. (multiplicity of infection) on a suitable bacterial host (e.g. E. coli XL-1 blue or BL21 (DE3) pLysE) so as to produce a high density of plaques. Typically about 1 million such plaques must be obtained for a fully representative sampling of cDNA species. The cDNA insert in such cloning vectors is typically under the control of a Lac I (Lac operon repressor) repressible promoter (e.g., that provided by the lac operator). Following the formation of lytic plaques (e.g. typically requiring incubation for 8 hours at 37° C., nitrocellulose filters which have been presoaked in 10 mM IPTG are overlayed on the plates. The IPTG induces expression of the cDNA species encoded by the phage lambda under the control of the lac promoter. The resulting plates are then incubated an additional 12-16 hours at 37° C., and the nitrocellulose filters are removed and blocked in 5% nonfat dry mild in TTBS (Blotto) for 2-16 hours at room temperature (r.t.) with gentle shaking. The blocked filters are then exposed to the Ub- or Ub1-trap probe.

A number of methods for labeling the material of the Ub- or Ub1-trap for use in Far-Western screening techniques exist in the art. Suitable TAGs for labeling the Ub- or Ub1-trap include antibody epitope tags (e.g. HA, FLAG, etc.), and biotin. Some of the alternative TAGs and methods for operably linking them to a Ub or Ub1 are described above.

In a preferred embodiment, Ub or Ub1 is used as the probe and the Ub- or Ub1-trap probe is synthesized by in vitro transcription and translation techniques which are well known in the art and available as kits from a number of sources (e.g. Promega Biotech, Madison, Wis.). Synthesis of the Ub or Ub1 molecule from a suitable Ub or Ub1 encoding vector in the presence of “S-met results in the synthesis of a ³⁵S-labeled Ub or Ub1 probe. The blocked filters produced as described above are then incubated in the presence of the ³⁵S-labeled Ub or Ub1 probe in fresh Blotto (typically 2 ml/1 50 nim filter) with gentle agitation overnight at room temperature or 4° C. The invention further provides that the incubation is also in the presence of a ubiquitination machinery of choice. The filters are then washed extensively with large volumes of TTBS several times to remove unbound Ub or Ub1 probe and then dried and exposed to X-ray film overnight. Plaques which appear to be labeled by the probe by virtue of an affinity between the gene product encoded by the cDNA and the Ub or Ub1 probe only in the presence of a ubiquitination machinery of choice, are picked and subjected to several rounds of plaque purification which involves the use of the above described procedure at increasingly low plating densities so as to facilitate the removal of contaminating plaques.

In a preferred embodiment of this Ub- or Ub1-trap Far-Western protocol, lysates are prepared from these pure clones and phage from these are used to infect a fresh culture of E. coli which are then incubated in the presence of 1 mM IPTG for 2 hours at 37° C. The cells are then lysed in SDS loading buffer, and the resulting lysate is run on a denaturing (e.g., SDS PAGE) protein gel along with dye labelled protein markers. The gel is transferred to nitrocellulose and probed with either the ³⁵S labeled Ub or Ub1 probe or a negative control probe in the presence or absence of a ubiquitination machinery of choice. The results of this type of Far-Western analysis thus reveal both the specificity of the interaction between the Ub or Ub1 probe and the lambda cDNA encoded candidate protein substrates (i.e., whether the ubiquitination occurs, as indicated by the presence of ³⁵S label, only with the Ub or Ub1 probe and not the negative control probe) and the molecular weight of the candidate protein substrate.

Equivalents: Those skilled in the art will recognize, or be able to ascertain using no more than routine experimentation, many equivalents of the specific embodiments of the invention described herein. Such equivalents are intended to be encompassed by the following claims.

The practice of embodiments of the present application will employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning: A Laboratory Manual, 2nd ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL press, 1986); B. Perbal, A Practical Guide to Molecular Cloning (1984); The Treatise, Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986).

Other features and advantages of the application will be apparent from the following detailed description, and from the claims.

References cited throughout this application are herein incorporated in full by reference.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

A yeast two-hybrid system to used for this invention is the GAL4-based MATCHMAKER™ system (BD Biosciences, Clontech). This system includes a plasmid termed pBridge that can be used to express the bait protein, for example, ubiquitin, fused the DNA-binding domain (DBD) of the GAL4 transcription activator. From the same plasmid to express the E3 protein from an inducible promoter MET25. This pBridge-ubiquitin/E3 plasmid will be used to screen a prey library of GAL4-activation domain (AD) fusion proteins, commercially available from BD Biosciences/Clontech and other suppliers or prepared by a skilled artisan. Similar protocols can be adapted using other yeast two-hybrid systems, for example, the LexA-responsive LacZ-based system.

Protocol 1:

a. Clone a copy of the Ub gene in frame with GAL4-BD in the plasmid pBridge to create pBridge-Ub.
b. Clone the gene (or a part of gene) of an E3 to be tested under the MET25 promoter of pBridge-Ub to create pBridge-Ub/E3.
c. Transform pBridge-Ub/E3 into the yeast strain AH109 and select colonies on media lacking tryptophan.
d. Either mate AH109(pBridge-Ub/E3) with the yeast strain Y187 containing prey library or transform AH109(pBridge-Ub/E3) with a DNA prey library.
e. Select diploid yeast or transformants on media lacking tryptophan, leucine, histidine, methionine and containing 3-amino triazole at varying concentrations (usually between 1 and 10 mM).
f. Isolate colonies that grow on this media and test them for growth on a similar media supplemented with methionine (1 mM) to suppress the expression of the E3.
g. Analyze colonies that grow on media lacking tryptophan, leucine, histidine, methionine but do not grow on media lacking tryptophan, leucine, histidine and containing methionine to identify gene in library plasmid.
   Protocol 2:
a. Clone a copy of the Ub gene in frame with GAL4-BD in the plasmid pBridge to create pBridge-Ub.
b. Clone the gene (or a part of gene) of an E3 to be tested under the MET25 promoter of pBridge-Ub to create pBridge-Ub/E3.
c. Transform pBridge-Ub/E3 into the yeast strain AH109 and select colonies on media lacking tryptophan.
d. Either mate AH109(pBridge-Ub/E3) with the yeast strain Y187 containing a prey plasmid isolated from a previous screen performed with a E3 bait or transform AH109(pBridge-Ub/E3) with such prey plasmid.
e. Select diploid yeast or transformants on media lacking tryptophan, leucine, histidine, methionine and containing 3-amino triazole at varying concentrations (usually between 1 and 10 mM).
f. Score colonies for growth on the same media and same media supplemented with methionine (1 mM) to suppress the expression of the E3 protein.
   Protocol 3:
a. Clone a copy of the Ub gene in frame with GAL4-BD in the plasmid pBridge to create pBridge-Ub.
b. Clone a copy of a “ubiquitination substrate” gene in frame with the GAL-AD in the plasmid pGADT7 (or similar) to create a plasmid pGAD-Substrate.
c. Transform both plasmids into yeast strain AH109 (or similar) and select colonies on media lacking tryptophan leucine.
d. Test that the transformants do not grow on media lacking tryptophan, leucine, histidine, methionine and containing 3-amino triazole at varying concentrations (usually between 1 and 10 mM). If transformants do not grow on this media the yeast can be used to screen cDNA library (step f.). If transformants grow on this media, see sub-protocol 3a bellow).
e. Prepare a cDNA yeast expression library under the MET25 promoter of pBridge-Ub.
f. Either mate AH109(pGAD-Substrate) with the yeast strain Y187 containing above library or transform AH109(pGAD-Substrate) with DNA of above library.
g. Select diploid yeast or transformants on media lacking tryptophan, leucine, histidine, methionine and containing 3-amino triazole at varying concentrations (usually between 1 and 10 mM).
h. Isolate colonies that grow on this media and test them for growth on a similar media supplemented with methionine (1 mM) to suppress the expression of the E3 protein.
i. Analyze colonies that grow on media lacking tryptophan, leucine, histidine, methionine but do not grow on media lacking tryptophan, leucine, histidine and containing methionine, to identify clone in library plasmid.
   Protocol 4:

When Ub bait and prey/substrate show positive interaction in the absence of added E3 expression plasmid two option should be distinguished. Either there is non-covalent interaction between ubiquitin and tested “ubiquitination substrate” or one of the yeast E3 proteins is a functional ligase for this substrate.
a. Perform immunoprecipitation of “ubiquitination-substrate” isolated from cells from step d. of protocol 3 under denaturing conditions. Separate immunoprecipitate by SDS-PAGE and immunodetect with antibodies against GAL4BD and in parallel with antibodies against the “ubiquitination substrate.” If there is a band that reacts with both antibodies and is of apparent molecular weight that is in accordance with the combined molecular weight of both proteins than there is ubiquitination that is carried out by a yeast E3 protein. If such a band is not observed the interaction is a non-covalent.
b. If it was found that ubiquitination is carried out by a yeast E3, this yeast E3 can be identified by using a panel of yeast strains each one caring a deletion of a different E3 (the yeast Saccharomyces cerevisiae has only 50-75 different E3 proteins).
c. Identifying said yeast E3 might give information about a subset of mammalian E3s that are likely to be the natural E3 of said substrate and will allow to perform screen (steps f. to i. in protocol 3) in the yeast strain deleted for said E3 ligase.

### Example 2

A N-terminal fusion of Ub to the DBD in pBridge was made. A POSH with the RING domain and a POSH without the RING domain were also cloned into the pBridge/DBD-Ub (DBD-Ub/POSH and DBD-Ub/POSHΔRING, respectively). In a yeast two-hybrid screening, the DBD-Ub/POSH bait fusion protein resulted in about 50 times more positive clones than the DBD-Ub/POSHΔRING, indicating:
1) DBD-Ub can be used by POSH to modify a substrate;
2) RING confers substrate recognition and/or ligase activities in this screening system; and
3) the number of protein substrates that can be ubiquitinated by a POSH-mediated ubiquitination machinery is far greater than the number of proteins that may interact with Ub independent of a POSH-mediated ubiquitination machinery.

1-21. (canceled)

22. A method for identifying a candidate E3 protein that acts on a protein substrate, comprising:
i. providing a host cell comprising:
a) a first nucleic acid encoding said candidate E3 protein,
b) a second nucleic acid encoding a bait fusion protein comprising a bait polypeptide fused to a first output-inducing polypeptide; and
c) a third nucleic acid encoding a prey fusion protein comprising a prey polypeptide substrate fused to a second output-inducing polypeptide,
wherein said prey polypeptide substrate is known to be the substrate of an E3 protein, and wherein physical proximity of said first and second output-inducing polypeptides induces an output signal; and
ii. detecting said output signal;
wherein the presence of said output signal indicates that said candidate E3 protein acts as an E3 protein with respect to the prey polypeptide.

23. The method of claim 22 further comprising determining whether the presence of said output signal is dependent on the presence of said candidate E3 protein in said host cell, wherein the dependency indicates that said candidate E3 protein acts as an E3 protein with respect to the prey polypeptide.

24. The method of claim 22, wherein said bait polypeptide comprises ubiquitin or a fragment thereof.

25. The method of claim 22, wherein said bait polypeptide comprises a ubiquitin-like protein modifier or a fragment thereof.

26. A method for identifying a protein substrate for an E2 protein, comprising:
i. providing a host cell comprising:
1. a first nucleic acid encoding said E2 protein,
2. a second nucleic acid encoding a bait fusion protein comprising a bait polypeptide fused to a first output-inducing polypeptide; and
3. a third nucleic acid encoding a prey fusion protein comprising a prey E3 polypeptide fused to a second output-inducing polypeptide,
wherein physical proximity of said first and second output-inducing polypeptides induces an output signal; and
ii. detecting said output signal;
wherein the presence of said output signal indicates that said prey E3 polypeptide comprises a candidate protein substrate for said E2 protein.

27. A method for identifying a candidate E2 protein that acts on an E3 protein substrate, comprising:
i. providing a host cell comprising:
1. a first nucleic acid encoding said candidate E2 protein,
2. a second nucleic acid encoding a bait fusion protein comprising a bait polypeptide fused to a first output-inducing polypeptide; and
3. a third nucleic acid encoding a prey fusion protein comprising a prey E3 polypeptide substrate fused to a second output-inducing polypeptide,
wherein physical proximity of said first and second output-inducing polypeptides induces an output signal; and
ii. detecting said output signal;
wherein the presence of said output signal indicates that said candidate E2 protein acts as an E2 protein with respect to the prey E3 polypeptide.

28. A host cell comprising:
i. a first nucleic acid encoding an E2 protein,
ii. a second nucleic acid encoding a bait fusion protein comprising a bait polypeptide sequence fused to a first output-inducing polypeptide; and
iii. a third nucleic acid encoding a prey fusion protein comprising a prey E3 polypeptide sequence fused to a second output-inducing polypeptide,
wherein physical proximity of said first and second output-inducing polypeptides induces an output signal.

29. The host cell of claim 28 further comprising a nucleic acid encoding an exogenous E1 protein.

30. A method for determining whether a first polypeptide mediates the attachment of a ubiquitin or ubiquitin-like protein to a prey polypeptide, the method comprising:
i. providing a host cell comprising:
1. a first nucleic acid encoding said first polypeptide,
2. a second nucleic acid encoding a bait fusion protein comprising a bait polypeptide fused to a first output-inducing polypeptide; and
3. a third nucleic acid encoding a prey fusion protein comprising a prey polypeptide fused to a second output-inducing polypeptide,
wherein said bait polypeptide comprises a ubiquitin or ubiquitin-like protein, or a fragment thereof sufficient to achieve covalent attachment to a suitable prey polypeptide, wherein said first polypeptide mediates covalent attachment of the bait polypeptide to a prey polypeptide that is a protein substrate of said first polypeptide, and wherein said first and second output-inducing polypeptides generate an output signal when the bait fusion protein interacts with the prey fusion protein; and
ii. detecting said output signal;
wherein the presence of said output signal indicates that said first polypeptide mediates the attachment of a ubiquitin or ubiquitin-like protein to said prey polypeptide.

31. The method of claim 30 further comprising determining whether the presence of said output signal is dependent on the presence of said first protein in said host cell, wherein the dependency indicates said first polypeptide mediates the attachment of a ubiquitin or ubiquitin-like protein to said prey polypeptide.

32. The method of claim 30, wherein said first polypeptide is an E3 protein, or active portion thereof.

33. The method of claim 32, wherein said prey polypeptide is a protein that is known or suspected to form a covalent bond with a ubiquitin or ubiquitin like protein.

34. The method of claim 30, wherein said first polypeptide is an E2 protein, or active portion thereof.

35. The method of claim 34, wherein said prey polypeptide is an E3 protein, or portion thereof, that is known or suspected to form a covalent bond with a ubiquitin or ubiquitin like protein.

36. The method of claim 30, wherein said first output-inducing peptide comprises a DNA binding domain of a transcriptional activator and said second output-inducing peptide comprises an activation domain of a transcriptional activator.

37. The method of claim 36, wherein said output signal is the expression of a reporter gene that is activated by said transcriptional activator.

38. The method of claim 30, wherein said output signal is a change in fluorescence.

39. The method of claim 37, wherein said reporter gene is endogenous to said host cell.

40. The method of claim 37, wherein said reporter gene is encoded by an expression construct exogenous to said host cell.

41. The method of claim 30, wherein expression of said first polypeptide is controlled by an inducible promoter.

42. The method of claim 30, wherein said host cell further comprises a fourth nucleic acid encoding an exogenous E1 protein.
